(19) United States
(12) Patent Application Publication
(10) Pub. No.: US 2014/0222093 A1
(43) Pub. Date: Aug. 7, 2014

(54) BONE REDUCTION DEVICE HAVING RO MARKERS AND METHOD OF USING THE SAME

(71) Applicant: KYPHON SARL, Neuchatel (CH)

(72) Inventor: Masoumeh Mafi, Mountain View, CA (US)

(73) Assignee: KYPHON SARL, Neuchatel (CH)

(21) Appl. No.: 13/760,132

(22) Filed: Feb. 6, 2013

(51) Int. Cl. A61B 17/88

(52) U.S. Cl. CPC A61B 17/8855
USPC 606/86 R

(57) ABSTRACT

A device and method for treating bone fractures/lesions using an inflatable body is provided. The inflatable body has a substantially flat horizontal surface for quick easy insertion into bone beneath the fracture so as to align misaligned fragments of the fracture and/or to collapsed bone. The substantially flat horizontal surface having a radio-opaque material has a higher durometer (less elastic), along the balloon web, than the rest of the inflatable body providing for optimum compression of the bone marrow.

### TECHNICAL FIELD

The present disclosure relates to inflatable devices for the treatment of bone fractures.

### BACKGROUND

Fractures, lesions and collapsing of bone structure can occur in humans due to age, disease or trauma. There are many areas of bone that are prone to collapsing/depression, such as vertebra, proximal humerus, tibial plateau, distal radius and calcaneous. A bone tamp can be used to restore collapsed bone and re-align bone fragments caused by fractures followed by injection of bone cement to fill any fractures, as well as, the void created after the inflated device is removed. Precise positioning of the inflatable device beneath the deepest point of the depression in the collapsed area or bone fragments is essential in properly restoring the correct alignment of the bone fragments or reduction of the bone using a rounded inflatable device. If a round inflatable device is improperly placed beneath the depression, misalignment of the fragments or under/over reduction can result. Time and skill of the surgeon as well as limited work area to maneuver the device makes it difficult to always achieve proper placement. A better inflatable body that facilitates proper placement in bone is needed.

### SUMMARY

This application is directed to a bone reduction device and method for treating fractures/lesions in bone. In particular, a bone reduction device is provided. The bone reduction device comprises a fill tube extending along a longitudinal axis having a proximal end, a distal end, and a lumen extending from the proximal end to the oppositely disposed distal end along the longitudinal axis. An inflatable body having a wall configured to define a fillable cavity attached to the distal end of the fill tube. The fillable cavity is in fluid communication with the lumen of the fill tube and the inflatable body is configured to have at least one substantially flat horizontal surface when inflated. The wall of the inflatable body has a first wall portion comprising material having a high durometer of nonelastic material, such as PET, Nylon and PEEK or semi-elastic, such as polyurethane and PEBAX (54-75 Shore D) and a second wall portion comprising material having a lower durometer of semi-elastic material, such as polyurethane and PEBAX and silicone (50-94 Shore A) than the first wall portion. The higher durometer first wall portion comprises radio-opaque material, for example, radio-opaque markers and dye, so that the orientation of the higher durometer first wall portion inside a surgical site can be determined prior to inflation. When the inflatable body is inflated the higher durometer first wall portion expands less than the lower durometer second wall portion. The higher durometer first wall portion forms the substantially flat horizontal surface the location/position of which is defined by the location of the strategically positioned radio-opaque markers.

A method for treating a bone fracture, comprising preparing bone for receiving the bone reduction device of the present disclosure is provided. Inserting the device into the bone wherein the inflatable body is in a deflated state. Detecting the radio-opaque material using a medical imaging device indicating the position, location and length of the substantially flat horizontal surface of the higher durometer first wall portion so as to aid in the placement of the inflatable body to assure proper alignment of the first wall portion within the surgical site. Orientating the horizontal surface of the inflatable body below a fracture or collapsed portion of bone based on the position of the radio-opaque material using the medical imaging device. Inflating the inflatable body with, for example saline or compressed air, so that the substantially flat horizontal surface of the higher durometer first wall portion compacts calcaneous bone and/or bone marrow to create a cavity and establish zero malreduction of the bone. Deflating the inflatable body and removing the deflated inflatable body from the bone.

A bone reduction kit comprising, a catheter having a lumen along a longitudinal axis, a fill tube positioned within the catheter extending along a longitudinal axis is also provided. The fill tube having a proximal end, a distal end, and a lumen extending from the proximal end to the oppositely disposed distal end along the longitudinal axis. An inflatable body having a wall configured to define a fillable cavity attached to the distal end of the fill tube. The fillable cavity is in fluid communication with the lumen of the file tube. The inflatable body having a first wall portion comprising material having a higher durometer and a second wall portion comprising material having a lower durometer than the first wall portion so as to produce a substantially flat horizontal surface configured to engage bone and apply force against the bone when inflated. The first wall portion comprising a strip of radio-opaque markers strategically placed extending from a proximal end to a distal end along a longitudinal axis of the inflatable body so as to show the position/location of the flat surface when viewed in situ by imaging machinery. The kit also comprises at least one cannula. In one embodiment the kit may contain different sized inflatable devices as well as different sized cannulas. The device is configured for insertion through the cannula into the bone adjacent the fracture.

These and other embodiments are further described in the figures and Detailed Description Sections directly following.

### BRIEF DESCRIPTION OF THE DRAWINGS

The present disclosure will become more readily apparent from the specific description accompanied by the following drawings, in which:

FIG. 1 is a perspective view of one particular embodiment of a bone fracture reduction device in accordance with the principles of the present disclosure;

FIG. 1A is a cross section between the arrows indicated in FIG. 1showing the radio-opaque markers;

FIG. 1B is the device shown in FIG. 1A rotated clockwise 90 degrees about longitudinal axis L;

FIG. 2 is an anterior/posterior view of the bone fracture reduction device for use in a long bone such as the tibial plateau, constructed in accordance with the teachings of the present disclosure;

FIGS. 3-7 are x-ray images of the inflatable body having different orientations at the surgical site; and

FIG. 8 is a perspective view of the inflatable body having in an inflated state having a strip of radio-opaque markers.

Like reference numerals indicate similar parts throughout the figures.

### DETAILED DESCRIPTION

A device and method is described for treating bone fractures and/or bone collapse. For illustrative purposes, the apparatus and method shall be described in the context of treating fractures and restoring proper reduction of a collapsed portion of a bone.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, distal and proximal, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament and/or bone, repairing a fracture or break in bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The term “reduction” as used in this application refers to a medical procedure to restore a fracture or dislocation to the correct alignment. When a bone fractures, the fragments lose their alignment in the form of displacement or angulation. For the fractured bone to heal without any deformity the bony fragments must be re-aligned to their normal anatomical position. Orthopedic surgeons attempt to recreate the normal anatomy of the fractured bone by reduction.

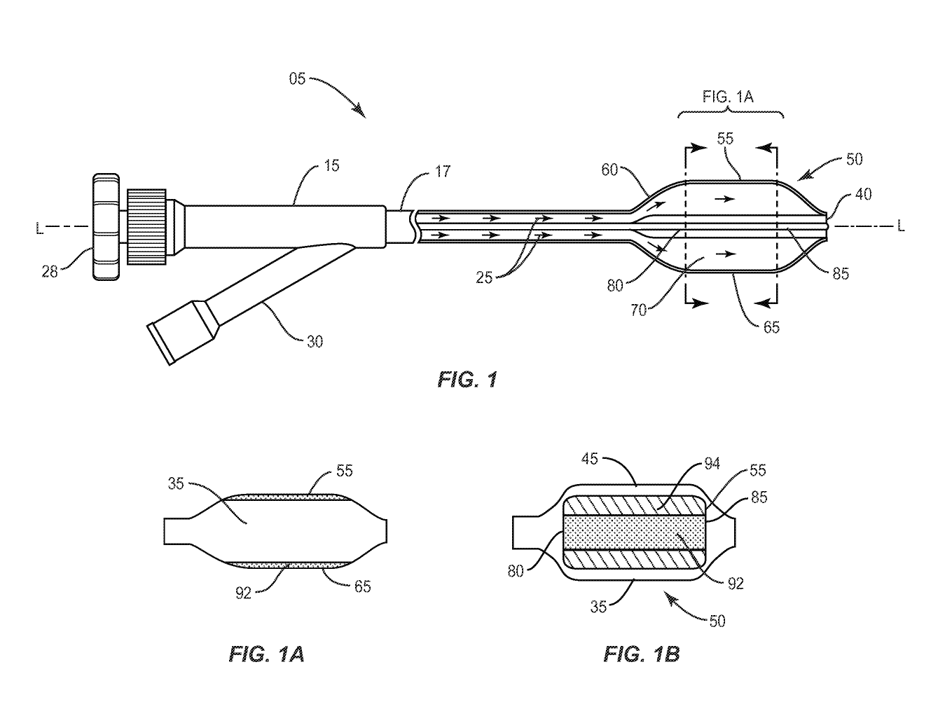

The following discussion includes a description of a device for treating bone lesions, fractures and/or collapsed bone and related methods of employing the device in accordance with the principles of the present disclosure. Alternate embodiments are also disclosed. Reference will now be made in detail to the exemplary embodiments of the present disclosure, which are illustrated in the accompanying figures. Turning now to FIGS. 1-8, there are illustrated components of the device for treating bone lesions, fractures and/or collapsed bone in accordance with the principles of the present disclosure.

The term “Touhy Borst” or “Y Tube” as used in the application refers to an adapter used for attaching catheters to various other devices.

The components of the bone reduction device can be fabricated from biologically acceptable materials suitable for medical apparatuses, including metals, synthetic polymers, ceramics, thermoplastic and polymeric material and/or their composites. For example, the components of the bone reduction device, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan, Fe—Mn—Si and Fe—Ni—Co—Ti composites), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers based materials, polymeric rubbers, polyolefin rubbers, semi-rigid and rigid materials, thermoplastic elastomers, thermoset elastomers, elastomeric composites, rigid polymers including polyphenylene, polyamide, polyimide, polyetherimide, polyethylene, epoxy, and composites of metals and calcium-based ceramics, composites of PEEK and calcium based ceramics, and combinations of the above materials. Various components of the anchoring bone reduction device may have material composites, including the above materials, to achieve various desired characteristics such as strength, rigidity, elasticity, compliance, and biomechanical performance, durability and to provide a non-stick surface. The components of the bone reduction device may be monolithically formed, extruded, coextruded, hot molded, cold molded, press molded, integrally connected or include fastening elements and/or coupling components, as described herein. In particular, the inflatable portion of the device according to the present disclosure can comprise flexible material, including (but not limited to) non-elastic materials such as PET, Mylar or Kevlar®, elastic materials such as polyurethane, latex or rubber, semi-elastic materials such as silicone, or other materials.

In particular, the higher durometer portion of the inflatable device can be made from one or the combination of materials, such as Nylon, PET, Polyurethane, PEBAX and PEEK. The combination of these materials and/or other materials used in the medical field creates the desired durometer characteristics needed for the device to work according to the teachings of the disclosures.

Since the inflatable body expands and comes in contact with cancellous bone, the materials used and thickness must have significant resistance so as to resist surface abrasion, puncture and/or tensile stresses. For example, structures incorporating elastomer materials, e.g., polyurethane, which have been preformed to a desired shape, e.g., by exposure to heat and pressure, can undergo controlled expansion and further distention in cancellous bone, without failure, while exhibiting resistance to surface abrasion and puncture when contacting cancellous bone. The present disclosure further discloses inflatable devices that have one or more biased directions of inflation. For example, inflatable devices having reduced lateral growth may provide improved fracture reduction because such devices can exert a greater vertical force and/or displacement within the treated bone. Such inflatable devices may also protect the lateral and anterior/posterior sidewalls of bone, for example long bone and vertebral body, by minimizing expansion towards these sidewalls and directing expansion to a greater degree along the longitudinal axis of the bone. In situations where a surgical procedure is terminated when the inflatable device contacts a lateral cortical wall of the targeted bone, such biased expansion could permit improved fracture reduction prior to reaching this procedure endpoint.

Radio-opaque markers can be made of tungsten powder, barium sulfate, gold or iridium. The radio-opaque markers should extend from the proximal end to the oppositely disposed distal end along the longitudinal axis.

As shown in FIG. 1, the reduction device 05, in accordance with the present disclosure, comprises a body 15 having a longitudinal axis “L” attached to a fill tube 17. Fill tube 17 extends from body 15 to the distal end 40 of device 05. The device 05 includes a lumen 25 extending from the proximal end 28 of body 15 to the oppositely disposed distal end 40 of device 05 along longitudinal axis L. Lumen 25 extends through fill tube 17 and is continuous with a fillable cavity 70 of an inflatable body, such as, for example, balloon 50. Fillable cavity 70 is defined by wall 60 and is configured to inflate upon receiving fluid material from fill tube 17. That is, fillable cavity 70 is attached to the distal end of the fill tube 17 and is in fluid communication with lumen 25 of fill tube 17 so that fluid material flowing from fill tube 17 inflates balloon 50. The fluid material used to inflate balloon 50 can be in the form of a liquid or gas, for example, saline, water or compressed air. Balloon 50 can be variously configured, such as, for example, unidirectional, omnidirectional or multiple lumens.

Wall 60 of balloon 50 has a first wall portion, such as, for example, first substantially flat horizontal surface 55 and a third wall portion, such as, for example, second substantially flat horizontal surface 65. First and second substantially flat horizontal surfaces 55, 65 extend from a proximal end 80 to a distal end 85 of balloon 50 along longitudinal axis L. First and second substantially flat horizontal surfaces 55, 65 are configured to engage bone and provide force against bone fragments so as to realign the bone fragments. First and second substantially flat horizontal surfaces 55, 65 are disposed on opposite sides of balloon 50 such that they are substantially parallel to one another. It is envisioned that first and second substantially flat horizontal surfaces 55, 65 are variously positioned with respect to one another, such as, for example, adjacent to one another or transverse to one another.

Balloon 50 has a second wall portion 35 and a fourth wall portion 45 extending from proximal end 80 to distal end 85 of balloon 50 along longitudinal axis L. When balloon 50 is inflated, second and fourth wall portions 35, 45 have a substantially circular or convex surface not configured to engage bone. Second and fourth wall portions 35, 45 are disposed on opposite sides of balloon 50 such that balloon 50 comprises a pattern of surfaces alternating between substantially flat horizontal and circular or convex. A Y tube 30 is connected to body 15 for attaching surgical devices as required by a particular procedure.

First and second substantially flat horizontal surfaces 55, 65 are made of material having a higher durometer, and are therefore less elastic, than second and fourth wall portions 35, 45. Second and fourth wall portions 35, 45 of outer wall 60 have a lower durometer reading, and are therefore more elastic, than first and second substantially flat horizontal surfaces 55, 65. The higher durometer first and second substantially flat horizontal surfaces 55, 65 prevents balloon 50 from fully expanding, therefore forming substantially flat horizontal surfaces 55, 65 of balloon 50. Second and fourth wall portions 35, 45 are made from material having a lower durometer than the rest of outer wall 60 and therefore will expand more than first and second substantially flat horizontal surfaces 55, 65, forming convex or arcuate surfaces, as shown in FIG. 1B.

The outer surface or inner surface of first and second substantially flat horizontal surface 55, 65 can contain ink, radio-opaque markings or fluorescence materials so that first and second substantially flat horizontal surfaces 55, 65 can be tracked when positioning balloon 50 in situ. It is envisioned that the radio-opaque markings, ink and/or fluorescence materials can be printed on an inner surface of first and second substantially flat horizontal surfaces 55, 65, or first and second substantially flat horizontal surfaces 55, 65 can be composed of the radio-opaque marks.

As shown in FIGS. 1A-1B, first and second substantially flat horizontal surfaces 55, 65 comprise a plurality of radio-opaque markers 92. Radio-opaque markers 92 are arranged as a strip or matrix 94 of a plurality of rows that extend along longitudinal axis L from a proximal end 80 to a distal end 85 of first and second substantially flat horizontal surfaces 55, 65 of balloon 50. It is envisioned that strip 94 of radio-opaque markers 92 extends across a central portion of first and second substantially flat horizontal surface 55, 65 (FIG. 1A) or may extend across an entirety of first and second substantially flat horizontal surfaces 55, 65. Strip 94 may be arranged in other patterns each in the goal of displaying the spatial orientation and positioning of first and second substantially flat horizontal surfaces 55, 65 so that the orientation of first and second substantially flat horizontal surfaces 55, 65 can be observed.

Strip 94 of radio-opaque markers 92 is about 1.5 mm by about 12 mm. In another embodiment of the present disclosure, the plurality of radio-opaque markers 92 may be in a plurality of rows that extend in an axis perpendicular to the longitudinal axis L across a width of first and second substantially flat horizontal surfaces 55, 65. Further, a side of fill tube 17 adjacent first substantially flat horizontal surface 55 can contain a plurality of radio-opaque markers to aid in the positioning of balloon 50.

Detecting radio-opaque markers 92 indicates the position, location and length of first and second substantially flat horizontal surfaces 55, 65 so as to aid in the placement of balloon 50 to assure proper alignment of fracture fragments when performing a procedure, for example, to fix collapsed bone. Radio-opaque markers 92 can be in any particular pattern on first and second substantially flat horizontal surfaces 55, 65 such that using a medical imaging device or fluoroscope when device 05 is inserted into bone, the surgeon will be able to determine not only the positioning of the substantially flat horizontal surfaces 55, 65 but also their orientation. This information allows for precise placement of the balloon 50, which often leads to better fracture repair. Any medical imaging technology can be used, such as radiography, fluoroscopy, luminescence, PET, SPECT, CT and MRI.

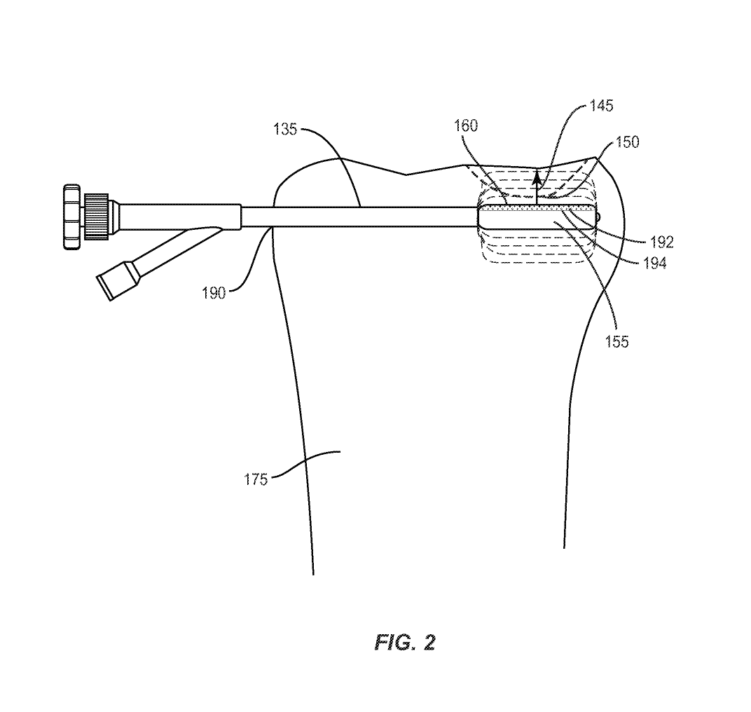

FIG. 2 shows the anterior/posterior view of a device 135 inserted into bone 175 at point 190. An inflatable body 155 having a substantially flat horizontal surface 160 is positioned directly beneath the deepest depression point 150 and is inflated in accordance with the present disclosure. In accordance with the teachings of the present disclosure, a region having radio-opaque markers 192 is used to properly position substantially flat horizontal surface 160 beneath deepest depression point 150 of the collapsed bone so that when inflated, the inflatable body 155 displaces along path 145 and presses against the deepest depression point 150 of the bone applying constant and equal pressure so as to reestablish the height of the collapsed bone. Expansion of the inflatable body 155 aligns fragments of bone caused as result of the fracture so as to facilitate proper healing of the fractured bone.

In realigning bone fragments of a fracture and/or re-establishing height to a collapsed bone, proper placement of the inflatable body 155 is essential. That is, to realign and correct collapsed bone, placing substantially flat horizontal surface 160 of the inflatable body 155 properly beneath the fracture or collapsed area is essential for a favorable outcome of the treatment. The substantially flat horizontal surface 160 of the device 135 allows for easy placement under the fracture and/or collapsed area of the bone so that upon inflation of inflatable body 155 the bone fragments are realigned and/or collapsed bone is corrected. In contrast, round or spherical shaped balloons (similar to the second and fourth wall portions 35, 45 of the present disclosure) are more difficult to properly place in situ due to the curvature of the balloon. If not positioned below the deepest point of the depression, once expanded the round portion of the inflatable body only partially restores the height of the collapsed bone. That is, the point directly under the outermost point of the round balloon is advance higher than the rest of the balloon therefore leading to incomplete correction of collapsed bone and/or misalignment of the bone fragments.

Thus, the inflatable body 155 of the present disclosure, having a matrix of radio-opaque markers 192 disposed on, within or as a part of the substantially flat horizontal surface 160 allows for proper placement of the inflatable body 155 prior to expansion. Proper placement is essential in treating fractures and collapsed bones, for example in extremities of a human. Accordingly, a printed matrix 194 of radio-opaque markers 192 in the shape of bands/rows within, on or as a part of the inflatable body 155 defines the boundaries of substantially flat horizontal surface 160 as well as the orientation of substantially flat horizontal surface 160 allowing a surgeon to place the inflatable body 155 properly in situ therefore making it easier and less time consuming to achieve proper placement of the inflatable body 155 prior to inflation. This ultimately leads to a better outcome and less time in surgery for a patient.

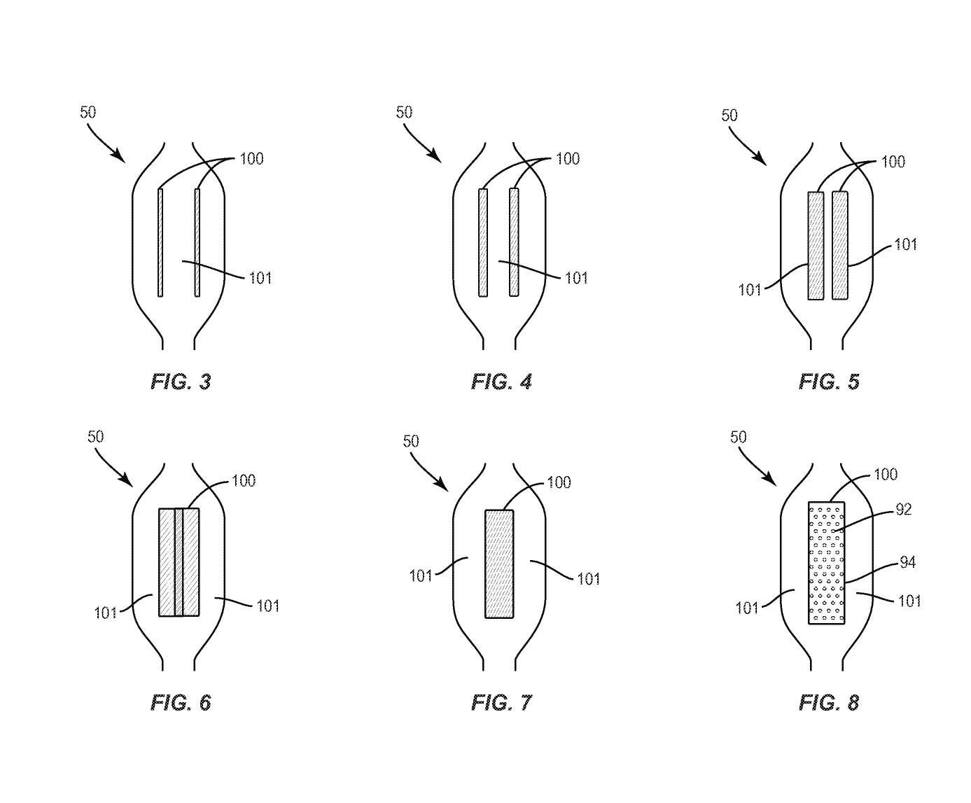

With reference to FIGS. 3-8, in assembly and use, the bone reduction device 05 described herein is employed with a surgical procedure for treatment of a disorder affecting a section of bone, such as a fracture, for example in a vertebrae or extremity of a patient. In use, the bone to be filled with balloon 50 is prepared to receive balloon 50 (such as by punching, drilling or otherwise displacing a small amount of the cancellous bone directly beyond the opening of the cannula). Balloon 50 is initially in a deflated state and the deflated balloon 50 is advanced into the bone through a cannula (not shown). The balloon 50 is oriented preferably in the bone such that substantially flat horizontal surfaces 55, 65 are positioned beneath the fracture. This is accomplished by using, for example, radio-opaque markers 92 which are disposed within and/or on first and second substantially flat horizontal surfaces 55, 65 in conjunction with medical imaging to effect orientation and placement in accordance with the description of the present disclosure. Using, for example, radiography to display the balloon 50 in a position within the patient's body, radio-opaque markers 92 will also be displayed.

FIG. 3 is an x-ray image displaying radiopaque-coated substantially flat horizontal surfaces 100 of the balloon 50 with a lower durometer convex wider side 101 of the balloon 50 positioned parallel to the x-ray beam. FIG. 4 is an x-ray image displaying the radiopaque-coated substantially flat horizontal surfaces 100 rotated clockwise approximately 20 degrees such that the radiopaque-coated substantially flat horizontal surfaces 100 appear closer together with the wider side 101 appearing to be closing. FIGS. 5-6 are the same images as displayed in FIGS. 3-4 except that the balloon has again been rotated clockwise showing that the radiopaque strips 94 are almost in complete coaxial alignment and appear to be adjacent one another and blurred. FIG. 7 is an x-ray image displaying the radiopaque-coated substantially flat horizontal surfaces 100 in complete coaxial alignment as evidenced by a solid black strip. The substantially flat horizontal surfaces 100 are perpendicularly positioned to the x-ray beam with the wider sides 101 extending in a plane parallel to the x-ray beam. An embodiment of the present disclosure provides that balloon 50 is rotated in either clockwise or counter-clockwise repeatedly and in smaller increments of rotation until the x-ray image displays the radio-opaque strips 100 in the desired orientation (e.g., solid dark strip as shown in FIG. 7).

Once the balloon 50 is orientated in the preferred orientation using the technique described above, filler material is directed into the finable cavity 70 of the balloon 50 so as to expand the balloon 50 and exert pressure on the cortical bone to realign fragments of the fracture and/or elevate collapsed bone to its proper height. (FIG. 2). Where such fracture or collapse has not occurred, such pressure would desirably compress the bone marrow and/or cancellous bone against the inner wall of the cortical bone, thereby compacting the bone marrow of the bone to be treated and to further enlarge the cavity in which the bone marrow is to be replaced by a biocompatible, flowable bone material, such as bone void filler/bone cement. The balloon 50 is then deflated and removed from the bone. In an embodiment of the present disclosure, bone filling material is injected into the cavity formed by the balloon once the balloon is removed so as to treat and/or maintain zero malreduction.

In a particular embodiment, elements of the bone reduction device may be included in a surgical kit. The kit may include a catheter having a lumen along a longitudinal axis; a fill tube positioned within the catheter extending along a longitudinal axis, the fill tube having a proximal end, a distal end, and a lumen extending from the proximal end to the oppositely disposed distal end along the longitudinal axis; an inflatable body having a wall configured to define a fillable cavity attached to the distal end of the fill tube wherein the fillable cavity is in fluid communication with the lumen of the fill tube. The inflatable body has a first wall portion comprising material having a higher durometer and a second wall portion comprising material having a lower durometer than the first wall portion so as to produce a substantially flat horizontal surface configured to engage bone and apply force against the bone when inflated. The first wall portion comprises a strip of radio-opaque markers extending from a proximal end to a distal end along a longitudinal axis of the inflatable body; and a cannula. In general, one or more of the above elements or any of the components required to carry out the method of the present disclosure may be stored in a sterilized package together. For example, the catheter, fill tube, inflatable body and cannula may be housed within a sterilized package, as well as bone filler.

Overall, the present disclosure includes a single or multi-lumen balloon that creates a substantially flat horizontal surface (pancake or wedge shaped) as it inflates. This will minimize the need for precision in balloon placement. It also eliminates the need for inflating multiple balloons in order to achieve adequate fracture reduction. This balloon gives surgeons a tool that simulates a bone tamp, while at the same time providing better control and a broader contact surface with the depressed area. Thus, it distributes the force uniformly across the depressed area. The orientation of the balloon in relation to the fracture fragments is critical. The current disclosure facilitates positioning of the flat region (wider side) of the balloon under the fluoro images. The present disclosure provides for printed matrices of dots in the shape of bands on the balloon stripes where expansion is minimal during inflation (the substantially flat horizontal portion). The stripes are, for example, extruded along the internal web of the balloon (multi-lumen extrusion). The stripes are made of a higher durometer material, which are centered along the longitudinal length of the balloon and inflation/expansion of the balloon will occur at both sides of the stripes. The radio-opaque mark matrix region can be printed on the stripes after the balloon forming. The printed patterns of radio-opaque dots can be in the form of a regular geometric matrix. The patterns are printed in the form of bands/rows of dots on the stripe region of the balloon with the total width and length being about 1.5 mm by 12 mm, with these dimensions being variable. The bands are positioned on the stripes, which are centered along the working length of the balloon. The stripe region of the balloon is made of a higher durometer material so as to expand less than the non-striped region of the balloon. The surface of the stripes can be chemically or plasma treated for more robust bonding between the radio-opaque markers and balloon material. The present disclosure makes it possible to distinguish the orientation of the balloon in different planes and recognize when the balloon is precisely horizontal or vertical to an x-ray beam. Thus, it is possible to directly detect the position/orientation of the balloon before the balloon is inflated.

As discussed above, spherical portions of the inflatable body may be restrained by using inelastic, semi-elastic, elastic and elastomeric materials in the construction of portions of the inflatable body. The material of the inflatable body can be a non-elastic material, such as polyethylene terephthalate (PET), nylon, Kevlar® or other medical inflatable body/balloon materials. It can also be made of semi-elastic materials, such as silicone, rubber, thermoplastic rubbers and elastomers or elastic materials such as latex or polyurethane. The higher durometer material wall portions having a higher young's modulus can be continuous or made of discrete elements of a flexible, inelastic high tensile strength material having the same or different durometer and thicknesses. The thickness of the inflatable body is typically in the range of 2/1000ths to 25/1000ths of an inch, although other thicknesses that can withstand increased pressures, such as 250-400 psi or greater, even up to 500, 1000 or 2000 psi, may be used.

Since the inflatable body expands and comes in contact with cancellous bone, the materials used and thickness of the wall of the inflatable body must have significant resistance so as to resist surface abrasion, puncture and/or tensile stresses. For example, structures incorporating elastomer materials, e.g., polyurethane, which have been preformed to a desired shape, e.g., by exposure to heat and pressure, can undergo controlled expansion and further distention in cancellous bone, without failure, while exhibiting resistance to surface abrasion and puncture when contacting cancellous bone. The present disclosure further discloses inflatable devices that have one or more biased directions of inflation. For example, inflatable devices having reduced lateral growth may provide improved fracture reduction because such devices can exert a greater vertical force and/or displacement within the treated bone. Such inflatable devices may also protect the lateral and anterior/posterior sidewalls of the vertebral body by minimizing expansion towards these sidewalls and directing expansion to a greater degree along the longitudinal axis of the spine. In situations where a surgical procedure is terminated when the inflatable device contacts a lateral cortical wall of the targeted bone, such biased expansion could permit improved fracture reduction prior to reaching this procedure endpoint.

The present disclosure further discloses inflatable devices having biased inflation along the longitudinal axis of the inflatable devices. Inflatable devices capable of biased inflation along their longitudinal axes may provide improved fracture reduction as such devices can be preferentially expanded towards areas of higher cancellous bone density and/or away from areas of lower cancellous bone density. Similarly, inflatable devices capable of biased inflation along their longitudinal axes can be preferentially expanded towards areas that resist expansion of the device and/or away from areas that promote expansion of the device.

Due to the nature of the injury, disease or other treatments, as well as the health and age of the patient suffering from these injuries, it may be preferable to treat a bone with the devices of this disclosure during an open or semi-open surgical procedure. In addition, a goal of the surgery may be to replace the diseased or injured bone with materials (such as bone fillers or certain drugs) which do not flow, and which thus are not well suited for a more minimally invasive procedure.

It should be understood that the various embodiments of inflatable body disclosed herein are by no means limited in their utility to use in a single treatment location within the body. Rather, while exemplary treatment location, these embodiments can be utilized in various locations within the human body are provided; this disclosure should not be so limited. For example, the device according to the present disclosure can be useful in treating a fractures in various other areas within the body, including but not limited to fractures and/or impending fractures of the femur, the radius, the ulna, the tibia, the humerus, or the spine. Similarly, the various other disclosed embodiments can be utilized throughout the body, with varying results depending upon treatment goals and/or the anatomy of the targeted bone.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone reduction device, comprising:
a fill tube extending along a longitudinal axis having a proximal end, a distal end, and a lumen extending from the proximal end to the oppositely disposed distal end along the longitudinal axis;
an inflatable body having a wall configured to define a fillable cavity attached to the distal end of the fill tube wherein the fillable cavity is in fluid communication with the lumen of the fill tube and the inflatable body is configured to have at least one substantially flat horizontal surface when inflated, wherein the wall of the inflatable body has a first wall portion comprising material having a higher durometer and a second wall portion comprising material having a lower durometer than the first wall portion, said higher durometer first wall portion comprising radio-opaque material so that the orientation of the higher durometer first wall portion can be determined inside a surgical site prior to inflation, wherein when the inflatable body is inflated the higher durometer first wall portion expands less than the lower durometer second wall portion, said higher durometer first wall portion forming the substantially flat horizontal surface.

2. A bone reduction device according to claim 1, wherein the radio-opaque material comprises a plurality of radio-opaque markers.

3. A bone reduction device according to claim 2, wherein the plurality of radio-opaque markers comprises a matrix of the plurality of radio-opaque markers.

4. A bone reduction device according to claim 3, wherein the matrix of the plurality of radio-opaque markers comprises a plurality of rows extending along a longitudinal axis from a proximal end to a distal end of the inflatable body.

5. A bone reduction device according to claim 3, wherein the matrix of the plurality of radio-opaque markers comprises a plurality of rows of radio-opaque markers extending along a longitudinal axis from a proximal end to a distal end of the higher durometer first wall portion.

6. A bone reduction device according to claim 5, wherein the plurality of rows of radio-opaque markers is printed on at least one of an outer surface and an inner surface of the higher durometer first wall portion.

7. A bone reduction device according to claim 3, wherein the plurality of rows of radio-opaque markers extend across a width of the higher durometer first wall portion.

8. A bone reduction device according to claim 3, wherein the matrix of the plurality of radio-opaque markers is about 1.5 mm by about 12 mm.

9. A bone reduction device according to claim 1, wherein the higher durometer first wall portion is at least one of fibrous material, woven material, polymeric material and non-elastic material.

10. A bone reduction device according to claim 1, wherein the inflatable body comprises a balloon.

11. A bone reduction device according to claim 10, wherein the balloon is unidirectional, omnidirectional or multiple-lumen.

12. A bone reduction device according to claim 1, wherein the inflatable body comprises a third wall portion adjacent said second wall portion and comprising the same higher durometer as the first wall portion so as to form a substantially flat horizontal surface substantially parallel with and opposing said first wall portion; and a fourth wall portion adjacent said first wall portion, substantially parallel with and opposing said second wall portion and comprising the same lower durometer as the second wall portion.

13. A bone reduction device according to claim 1, wherein at least one side of said fill tube comprises a plurality of radio-opaque markers extending from the proximal end to the oppositely disposed distal end along the longitudinal axis.

14. A method for treating a bone fracture, comprising: preparing bone for receiving the bone reduction device of claim 1; inserting the device into the bone wherein the inflatable body is in a deflated state;
detecting the radio-opaque material using a medical imaging device indicating the position, location and length of the substantially flat horizontal surface of the higher durometer first wall portion so as to aid in the placement of the inflatable body to assure proper alignment of the first wall portion within the surgical site; orienting the substantially flat horizontal surface of the inflatable body below a fracture or collapsed portion of bone based on the position of the radio-opaque material using the medical imaging device; inflating the inflatable body so that the substantially flat horizontal surface of the higher durometer first wall portion compacts calcaneous bone and/or bone marrow to create a cavity and establish zero malreduction of the bone; deflating the inflatable body; and
removing the deflated inflatable body from the bone.

15. A method for treating a bone fracture of claim 14, wherein the steps of detecting the radio-opaque material using a medical imaging device and orientating the substantially flat horizontal surface of the inflatable body are repeated until the radio-opaque material of the higher durometer first wall portion and radio-opaque material of a higher durometer third wall portion are in substantial alignment in relation to an x-ray beam, wherein the higher durometer third wall portion is adjacent said second wall portion and substantially parallel with and opposing said first wall portion.

16. A method for treating a bone fracture of claim 14, further comprising injecting a bone filling material into the cavity formed by the inflatable body once the inflatable body is removed so as to treat and/or maintain zero malreduction.

17. A method for treating a bone fracture of claim 14, wherein the medical imaging device used is at least one of radiography, fluoroscopy, luminescence, PET, SPECT, CT and MRI.

18. A method for treating a bone fracture of claim 14, wherein the radio-opaque material comprises a matrix of a plurality of radio-opaque markers.

19. A method for treating a bone fracture of claim 17, wherein the matrix of the plurality of radio-opaque markers comprises a plurality of rows of radio-opaque markers extending along a longitudinal axis from a proximal end to a distal end of the inflatable body.

20. A bone reduction kit, comprising:
a catheter having a lumen along a longitudinal axis; a fill tube positioned within the catheter extending along a longitudinal axis, the fill tube having a proximal end, a distal end, and a lumen extending from the proximal end to the oppositely disposed distal end along the longitudinal axis; an inflatable body having a wall configured to define a fillable cavity attached to the distal end of the fill tube wherein the fillable cavity is in fluid communication with the lumen of the fill tube, the inflatable body having a first wall portion comprising material having a higher durometer and a second wall portion comprising material having a lower durometer than the first wall portion so as to produce a substantially flat horizontal surface configured to engage bone and apply force against the bone when inflated, wherein the first wall portion comprises a matrix of radio-opaque markers extending from a proximal end to a distal end along a longitudinal axis of the inflatable body; and a cannula, wherein the device is configured for insertion through the cannula into the bone adjacent the fracture.
